(19) United States
(12) Patent Application Publication
(10) Pub. No.: US 2011/0189749 A1
(43) Pub. Date: Aug. 4, 2011

(54) TRAPPING, RECYCLING, AND OTHER TECHNIQUES INVOLVING CATALYSTS

(75) Inventors: Klavs F. Jensen, Lexington, MA (US); Lars Johansen, Kobenhavn (DK); Kevin Nagy, Somerville, MA (US)

(73) Assignee: Massachusetts Institute of Technology, Cambridge, MA (US)

(21) Appl. No.: 13/011,310

(22) Filed: Jan. 21, 2011

Related U.S. Application Data
(60) Provisional application No. 61/297,504, filed on Jan. 22, 2010.

(51) Int. Cl. C12N 11/00; B01L 3/00; B01J 19/00; F15D 1/00

(52) U.S. Cl. 435/174; 422/500; 422/129; 137/1

(57) ABSTRACT

Trapping, recycling, and other techniques involving catalysts are provided by this invention. The present invention provides for the retention of catalysts and other immobilized entities within a reaction region. In one aspect, the invention promotes such retention by incorporating support material regions including relatively little (or, in some cases, substantially no) catalyst (and thus, a relatively large number of catalyst adsorption sites) which can trap catalyst as it is transported through the downstream support material. In some cases, such arrangements can be achieved by using multiple beds arranged in series. In other instances, the amount of catalyst can be varied within a single bed to achieve the desired effect. The embodiments described herein can be used in systems in which the catalyst is covalently or non-covalently associated with the support surface.

### RELATED APPLICATIONS

This application claims priority under 35 U.S.C. §119(e) to U.S. Provisional Patent Application No. 61/297,504, filed Jan. 22, 2010, and entitled “Trapping, Recycling, and Other Techniques Involving Catalysts,” which is incorporated herein by reference in its entirety for all purposes.

### FIELD OF INVENTION

Trapping, recycling, and other techniques involving catalysts are generally described.

### BACKGROUND

Catalysts find widespread use in a variety of industries. The separation of the catalyst from the final product is often desirable. For example, in many pharmaceutical processes low levels of catalyst (e.g., heavy metals) must be maintained in the final product.

Separation of homogeneous catalysts from product streams can be difficult and expensive. Therefore, catalyst is often immobilized on a support that can be easily separated from the product stream. In many cases, including those in which reactions are performed in a fluid medium, the catalysts may leach from the support to the product stream, increasing the difficulty and cost of separation. Accordingly, systems and methods in which catalyst leaching is reduced and/or controlled would be desirable.

### SUMMARY OF THE INVENTION

The embodiments described herein are generally related to trapping, recycling, and other techniques involving catalysts. The subject matter of the present invention involves, in some cases, interrelated products, alternative solutions to a particular problem, and/or a plurality of different uses of one or more systems and/or articles.

In one aspect, a system is provided. In one set of embodiments, the system comprises a first portion of support material for immobilizing a catalyst, the first portion of support material and any catalyst thereon together defining a first volume, the first volume containing a catalyst at a first ratio of moles of catalyst to mass of the first portion of support material. The system can also comprise a second portion of support material for immobilizing a catalyst, the second portion of support material and any catalyst thereon together defining a second volume, the second portion of support material in fluid communication with the first portion of support material, and the second volume containing the catalyst at a second ratio of moles of catalyst to mass of the second portion of support material that is smaller than the first ratio.

In another aspect, a method is provided. In one set of embodiments, the method comprises establishing the flow of a fluid over a first portion of support material for immobilizing a catalyst, the first portion of support material and any catalyst thereon together defining a first volume having a first ratio of moles of catalyst to mass of support material. The method can also comprise subsequently establishing the flow of the fluid over a second portion of the support material in fluid communication with the first portion, the second portion of support material and any catalyst thereon together defining a second volume having a second ratio of moles of catalyst to mass of support material, wherein the second ratio is smaller than the first ratio.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1F include schematic illustrations of a system comprising multiple containers in which a catalyst is immobilized, according to one set of embodiments;

FIGS. 2A-2B include schematic illustrations of an exemplary system including a single container in which a catalyst is immobilized;

FIGS. 3A-3D include schematic illustrations of a system in which fluid flow across multiple containers is similar before and after the modification of flow, according to one set of embodiments;

FIGS. 4A-4B include schematic illustrations of (A) an exemplary catalyst comprising a metal and a ligand, and (B) an exemplary reaction scheme;

FIG. 5 includes a plot of conversion as a function of the number of residence times, according to one set of embodiments;

FIG. 6 includes a photograph of exemplary microreactors containing support material; and

FIG. 7 includes a photograph of microreactors containing catalyst and support material, according to one set of embodiments.

### DETAILED DESCRIPTION

Trapping, recycling, and other techniques involving catalysts and/or other immobilized entities are provided by this invention. In many applications, catalysts can be immobilized on a support material within a reactor. In some cases, the catalyst can be entrained in a fluid (e.g., a reactant) that is passed through a container (e.g., a reactor), which can lead to the undesirable transport of the catalyst out of the container (e.g., reactor). This can cause, in some cases, decreased reaction yields and conversion over time. In addition, inclusion of the immobilized entity in the final product stream can be undesirable in many instances (e.g., when the catalyst is toxic). The present invention provides for the retention of catalysts and other immobilized entities within a reaction volume or region. In one aspect, the invention promotes such retention by incorporating support material regions including relatively little (or, in some cases, substantially no) catalyst (and thus, a relatively large number of catalyst adsorption sites) which can trap catalyst as it is transported through the downstream support material. In some cases, such arrangements can be achieved by using multiple beds arranged in series. In other instances, the amount of catalyst can be varied within a single bed to achieve the desired effect. The embodiments described herein can be used in systems in which the catalyst is covalently or non-covalently associated with the support surface.

Advantageously, the embodiments described herein may allow for substantially continuous operation of a chemical reaction while simultaneously controlling catalyst transport (e.g., via trapping, recycling, etc.), allowing for substantially continuous product manufacture. In contrast, in many batch applications, recycling and/or trapping the catalyst can involve stopping the chemical reaction (sometimes for extended periods of time) to filter or otherwise isolate the support material. In addition, the embodiments described herein can allow for the use of relatively weakly physically-absorbed catalysts that might be otherwise impractical for use in systems in which catalyst retention is desirable. The use of physically-absorbed catalysts can be advantageous because, in many cases, physically absorbed catalysts are inexpensive, easy to synthesize, and/or easy to characterize relative to covalently bound analogues. Of course, the embodiments described herein can also be advantageous in systems that employ covalently-bound catalysts, providing a direct means by which leached catalysts (e.g., metals) are recycled.

The embodiments described herein may find application in a variety of fields. In some cases, the systems and methods can be used to control catalyst transport in fluid-based chemical reaction systems, at scales ranging from the microscale to large-scale systems. As another example, the embodiments described herein can be used to control catalyst transport in systems for screening catalysts for catalytic activity (e.g., high throughput catalyst screening systems). Examples of industries in which the embodiments described herein could be used include, for example, the pharmaceutical industry, the chemical and biochemical industries, the catalyst and fine materials industries, and the lab equipment industry, among others in which catalyst transport control is desired.

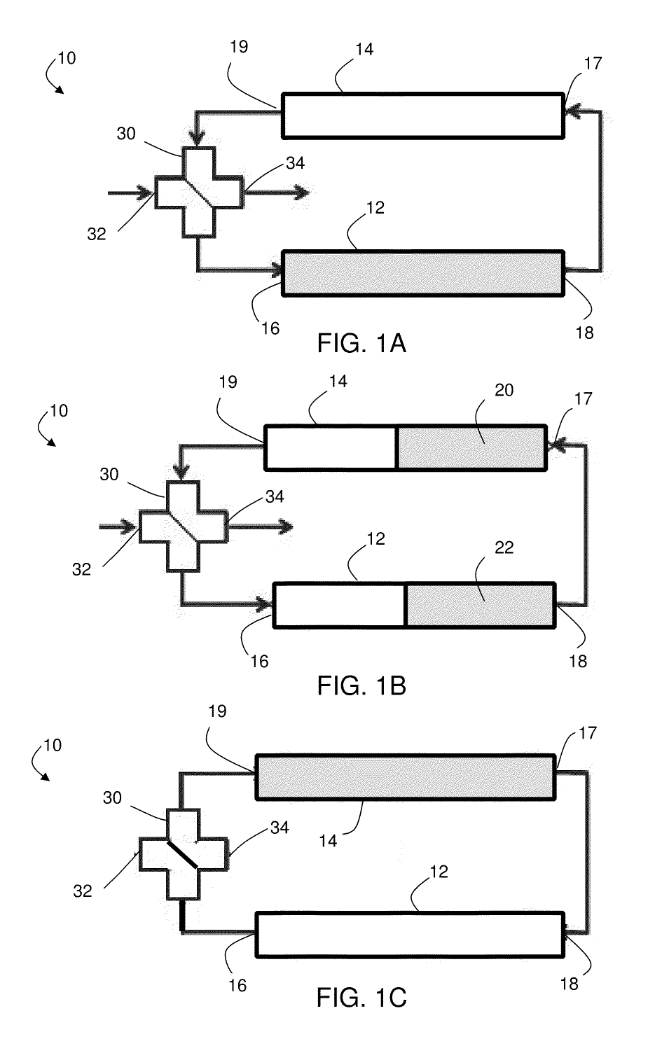

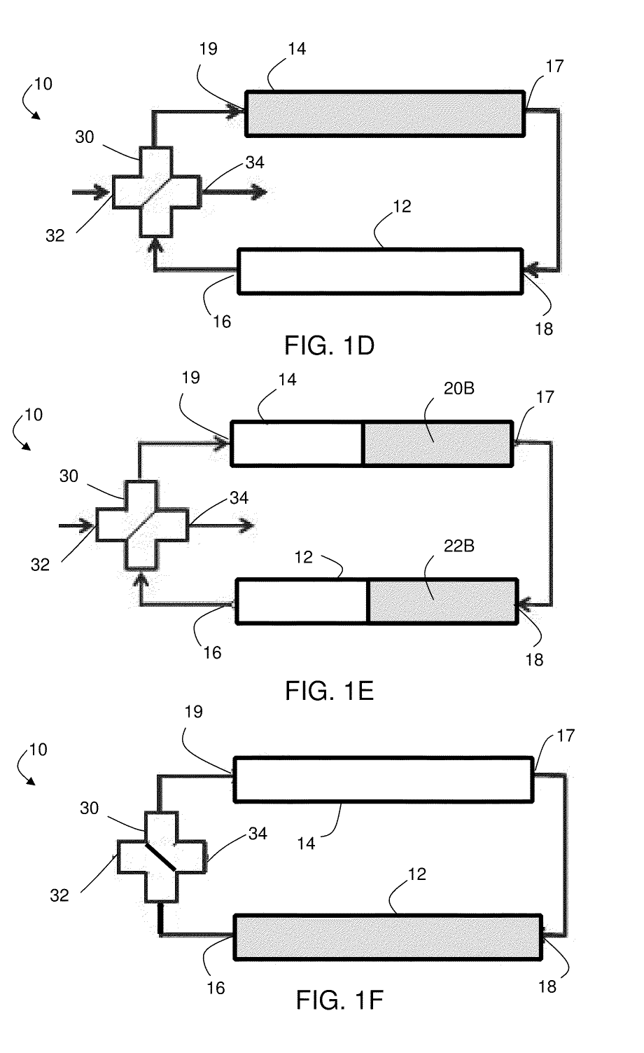

FIGS. 1A-1F include schematic illustrations of a system 10 in which an immobilized catalyst is transferred between volumes 12 and 14. The term “volume” is used generally to refer to any three-dimensional space, the boundaries of which are not necessarily defined by the walls of a container (e.g., a reactor). In FIGS. 1A-1F, first volume 12 includes a first portion of support material, and second volume 14 includes a second portion of support material. The support material in volumes 12 and 14 can, in some cases, be capable of immobilizing a catalyst. For example, in some cases, volumes 12 and/or 14 can be contained within a packed-bed (e.g., fixed-bed) reactor, for example, in which catalyst is immobilized on a support material. In some embodiments, a single type of support material can be contained within volumes 12 and/or 14, while in other embodiments, a mixture of multiple types of support material can be contained within volumes 12 and/or 14. In some embodiments, the support material within volumes 12 and 14 can be substantially similar in chemical and/or physical (e.g., size, shape, etc.) composition.

As shown in FIGS. 1A-1F, second volume 14 is in fluid communication with first volume 12. The term “fluid communication,” as used herein, refers to two volumes constructed and arranged such that a fluid can flow between them. In some cases, the first and second volumes can be in direct fluid communication. As used herein, two devices are in “direct fluid communication” when the fluidic connection between the two articles is uninterrupted by the presence of additional devices such as valves.

The first volume may contain, in some embodiments, catalyst immobilized on the support material within the first volume. While the first volume 12 in FIG. 1A includes catalyst substantially evenly distributed throughout the volume, it should be understood that the catalyst can be distributed within the volume in any suitable manner. For example, in some cases, the catalyst may be contained within a small region of the volume (e.g., as a pulse of catalyst near the inlet, etc.).

In some cases, the support material within the second volume (e.g., volume 14 in FIG. 1A) can contain available sites onto which catalyst can be immobilized. These available sites may allow for the immobilization of catalyst that is entrained within a fluid (containing, for example, a reactant and/or a reaction product) exiting the first volume (e.g., volume 12 in FIG. 1A) as that fluid is transported through the support material within the second volume, and a relative reduction of the amount of catalyst within the fluid stream exiting the second volume.

In some embodiments, the second volume can contain a relatively low ratio of catalyst to support material, relative to the ratio of catalyst to support material within the first volume. For example, the catalyst immobilized within the first volume can, in some cases, be present at a first ratio of moles of the catalyst to the mass of the support material within the first volume. The catalyst immobilized within the second volume can be present at a second ratio of moles of the catalyst to mass of the support material within the second volume, wherein the second ratio is smaller than the first ratio. Such comparisons between ratios can be made by dividing the first numbers of the ratios by the second numbers of the ratios, and comparing the resulting quotients. In some embodiments, the ratio of moles of catalyst to mass of support material within the second volume (i.e., the second ratio) can be at least about 5 times, at least about 10 times, at least about 100 times, at least about 1000 times, or at least about 10,000 times smaller than the ratio of moles of catalyst to mass of support material within the first volume (i.e., the first ratio). In some embodiments, catalyst can be immobilized on the support material defining the first volume while the second volume is substantially free of catalyst.

In some embodiments, the density of the catalyst in the second volume (mass of catalyst per unit volume) can be lower than the density of the catalyst in the first volume. In some such embodiments, as a fluid (which can include a reactant and/or a reaction product) is passed from the first volume to the second volume, the catalyst density within the first volume can decrease while the catalyst density within the second volume can increase.

In the set of embodiments illustrated in FIG. 1A, first volume 12 contains a support material and a catalyst immobilized on the support material, the presence of the catalyst indicated by the shading within first volume 12. Second volume 14 contains support material that is substantially free of catalyst, as indicated by the absence of shading within the volume. In some such embodiments, because the second volume contains a low amount of catalyst relative to the amount of support material within the second volume, a relatively large number of support sites are available within the second volume. Thus, catalyst that is transported out of the first volume can be immobilized by the relatively catalyst-deficient support material within the second volume.

The first and second volumes can be arranged in any suitable manner. In some embodiments, such as those illustrated in FIGS. 1A-1F, the two volumes may be part of separate containers that are connected by a line, a pipe, tubing, or some other suitable conduit. For example, the first volume may correspond to a first container (e.g., a microreactor, a test tube, etc.), and the second volume may correspond to a second container connected to the first container via tubing. In some cases, the first volume can be a sub-volume of a first container, and the second volume can be a sub-volume of a second container. In some embodiments, the use of separate containers can be advantageous, relative to the use of a single container. For example, the use of separate containers may allow for substantially independent control of the temperatures in the first and second volumes. In addition, the use of separate containers may make it easier to independently tailor surface chemistries within the first and second volumes.

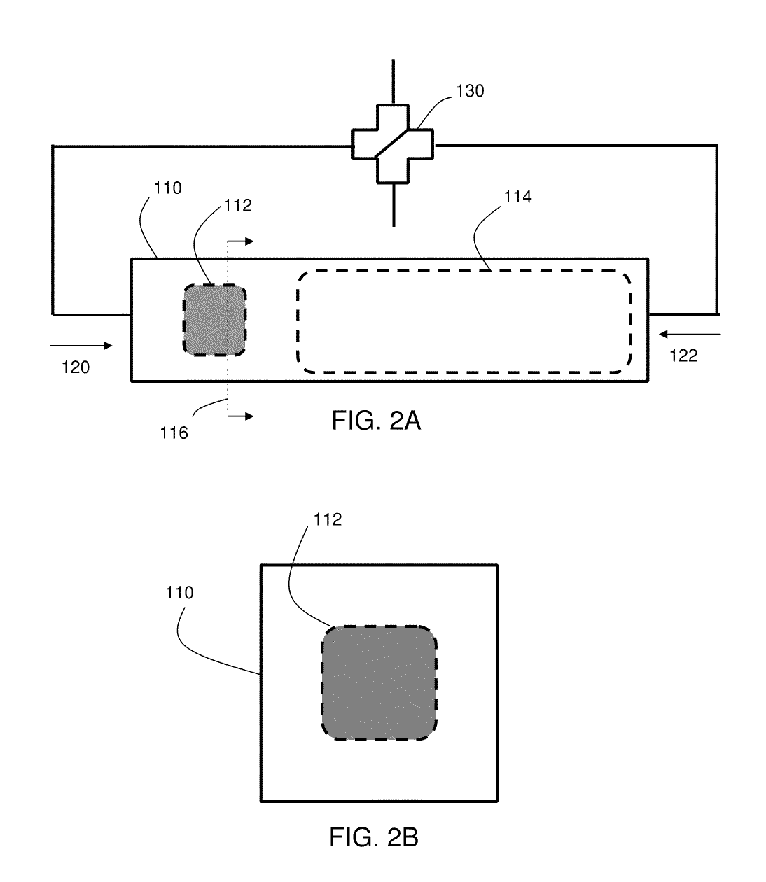

In some cases, the first and second volumes may be sub-volumes of a single, larger volume (e.g., of a single container). For example, in some embodiments such as those illustrated in FIG. 2A, the first volume may correspond to a first region of a container (e.g., region 112 of container 110 in FIG. 2A) while the second volume may correspond to a second, different region of the same container (e.g., region 114 in FIG. 2A).

In some cases, the first and/or second volumes may occupy at least a threshold percentage of a cross-sectional area of the container in which they are located. For example, in some cases, the first and/or second volumes occupy at least about 5%, at least about 10%, at least about 25%, at least about 50%, at least about 75%, at least about 90%, at least about 95%, at least about 99%, or substantially all of the area of a cross-section of a container in which the volume is located. As used herein, the “cross-section” of a container is measured between two opposed outer boundaries of the container. In some cases, the cross-section can be measured substantially perpendicular to the flow of fluid within the container. As a specific example, FIG. 2B includes a schematic illustration of a cross-section of container 110, as indicated by dashed line 116 in FIG. 2A. As shown in FIG. 2B, volume 112, which contains a catalyst immobilized on a support material, occupies about 20% of the cross-sectional area of container 110.

In some embodiments, a flow of fluid can be established through the first and second volumes. For example, a fluid containing one or more reactants can be flowed through the first and second volumes to perform a catalytic chemical and/or biological reaction. In some embodiments, the fluid can contain and liquid and/or gas. The fluid can contain an ionic liquid in some embodiments. In some embodiments, the fluid can include an inorganic component (e.g., water) and/or an organic component (e.g., hydrocarbons). In some cases, the fluid can contain a solvent. For example, the fluid can contain a solvent in which a reactant is dissolved.

As shown in FIG. 1A, a fluid can be transported into first volume 12 via interface 16 (which acts as an inlet in this case). Generally, the direction of fluid flow is indicated by arrows in FIGS. 1A-1F. As fluid is transported through the first volume, it may be flowed over the first portion of support material within the first volume. In some cases, at least a portion of the catalyst immobilized within the first volume may be transported out of the first volume. For example, in FIG. 1A, catalyst immobilized on the support material within first volume 12 can be entrained in the fluid transported through the first volume and exit the volume via interface 18 (which acts as an outlet in this case). Catalyst may be entrained in the fluid via any suitable mechanism. For example, in some cases, the catalyst may be leached from the support material (e.g., by dissolving in a solvent in the fluid).

In some embodiments, the fluid, which can contain the entrained catalyst from the first volume, can be transported to the second volume such that it is flowed over the support material within the second volume. At least a portion of the catalyst can, in some instances, be immobilized on the support material within the second volume as it is flowed over the support material. For example, FIG. 1B includes a schematic illustration outlining the distribution of catalyst after the fluid in which the catalyst becomes entrained is flowed over the second volume for a period of time. In FIG. 1B, the catalyst that had been entrained in the fluid as it was transported through first volume 12 is transported with the fluid into second volume 14 via interface 17 (which acts as an inlet in this case). A portion of the catalyst can be immobilized on the support material in second volume 14, as indicated by shaded region 20. The portion of the catalyst that remains within first volume 12 is indicated by shaded region 22. After the fluid is transported through the second volume, it can exit interface 19 (which acts as an outlet in this case) of second volume 14. It should be understood that the distributions of catalyst as illustrated in FIGS. 1A-1F are merely illustrative, and the actual distribution observed in practice may be different. For example, in some cases, the catalyst may be immobilized within the second volume such that the concentration of catalyst decreases along the length of the volume over a region longer than half the length of the volume.

In some embodiments, the adsorption of the catalyst onto the support material within the second volume can result in a relatively low amount of catalyst exiting the second volume, relative to the amount of catalyst that was present within the fluid upon entering the second volume. In some cases, at least about 50%, at least about 75%, at least about 90%, at least about 95%, at least about 98%, at least about 99%, at least about 99.9%, at least about 99.99%, or substantially all of the catalyst that exits the first volume can be immobilized within the support material within the second volume. In some cases, a relatively large amount of the catalyst originally immobilized within the first volume can be immobilized within the second volume. For example, in some instances, at least about 50%, at least about 75%, at least about 90%, at least about 95%, at least about 98%, at least about 99%, at least about 99.9%, at least about 99.99%, or substantially all of the catalyst that was originally immobilized within the first volume is immobilized within the second volume. In the set of embodiments illustrated in FIG. 1C, substantially all of the catalyst that was immobilized in first volume 12 of FIG. 1A has been immobilized within second volume 14.

In some cases, the flow of fluid may be altered such that fluid flows from the second volume to the first volume, rather than from the first volume to the second volume. For example, in some embodiments, when one or more valves within the system are in a first position, fluid (e.g., containing a reactant) from a source can be transported into a first volume and from the first volume into a second volume. The fluid flow can then be altered, for example, by adjusting the one or more valves (as described in more detail below) such that fluid is transported from the source into the second volume and from the second volume into the first volume.

Fluid flow can be altered, for example, after at least a portion of the catalyst originally immobilized within the first volume has been transported to and immobilized within the second volume. In some cases, fluid flow can be altered after at least about 50%, at least about 75%, at least about 90%, at least about 95%, at least about 98%, at least about 99%, at least about 99.9%, at least about 99.99%, or substantially all of the catalyst that was originally immobilized within the first volume has been transferred out of the first volume. In some cases, fluid flow can be altered after at least about 50%, at least about 75%, at least about 90%, at least about 95%, at least about 98%, at least about 99%, at least about 99.9%, at least about 99.99%, or substantially all of the catalyst that was originally immobilized within the first volume has been immobilized within the second volume.

In some cases, after altering the flow of the fluid, fluid can entrain at least a portion of the catalyst immobilized on the support material within the second volume. In some such embodiments, at least a portion of the catalyst immobilized within the second volume can be transported out of the second volume. For example, in FIG. 1D, fluid can be transported into second volume 14 via interface 19 (acting as an inlet in this case). In some cases, catalyst immobilized on the support material within second volume 14 can be entrained in the fluid transported through the second volume and exit the second volume via interface 17 (acting as an outlet in this case).

In some embodiments, after altering fluid flow, the fluid and the entrained catalyst exiting the second volume can be transported to the first volume such that the fluid and catalyst are flowed over the support material within the first volume. At least a portion of the catalyst can, in some instances, be immobilized on the support material within the first volume as it is flowed over the support material. For example, FIG. 1E includes a schematic illustration outlining the distribution of catalyst after fluid flow has been altered and the fluid has been transported over the first volume for a period of time. In FIG. 1E, that catalyst that was entrained in the fluid as it was transported through second volume 14 is transported with the fluid into first volume 12 via interface 18 (acting as an inlet in this case). A portion of the catalyst can be immobilized on the support material in first volume 12, as indicated by shaded region 22B. The portion of the catalyst that remains within second volume 14 is indicated by shaded region 20B in FIG. 1E. After the fluid is transported through the first volume, it can exit interface 16 (acting as an outlet in this case) of first volume 12.

In some embodiments, the adsorption of the catalyst onto the support material within the first volume can result in a relatively low amount of catalyst exiting the first volume, relative to the amount of catalyst that was present within the fluid at the inlet of the first volume (e.g., any of the amounts described above in relation to the amount of catalyst exiting the second volume). In some cases, a relatively large amount of the catalyst originally immobilized within the second volume can be immobilized within the first volume after flow has been altered (e.g., any of the amounts described above in relation to the amount of catalyst originally immobilized by the first volume subsequently immobilized by the second volume). In the set of embodiments illustrated in FIG. 1F, substantially all of the catalyst that was immobilized in second volume 14 of FIG. 1D has been immobilized within first volume 12.

The ability to alter the flow path of the fluid supplied to the first and second volumes can allow one to control the transport of catalyst within the system (e.g., by transferring catalyst back and forth between the first and second volumes). Flow may be altered any suitable number of times (e.g., at least 2 times, at least 3 times, at least 5, times, at least 10 times, at least 100 times, at least 1000 times). As such, catalyst can be transferred from a first volume to a second volume (and, optionally, back again) any suitable number of times (e.g., at least 2 times, at least 3 times, at least 5, times, at least 10 times, at least 100 times, at least 1000 times).

In some embodiments, the amount of catalyst that is transported out of the system defined by the first volume, the second volume, and any fluidic connections between the two volumes can be relatively low (e.g., less than about 20%, less than about 10%, less than about 5%, less than about 2%, less than about 1%, less than about 0.1%, less than about 0.01%, or less than about 0.001% of the total amount of catalyst originally contained within the first volume, the second volume, and any fluidic connections between the two volumes). In some cases, such small losses of catalyst from said system may be maintained over at least 5 flow cycles, at least 10 flow cycles, at least 100 flow cycles, at least 1000 flow cycles, at least 10,000 flow cycles, or more. A “flow cycle,” as used in this context, refers to a period of time commencing upon flow of the fluid (e.g., containing one or more reactants) from a first to a second volume, continuing through the point at which flow is altered such that the flow occurs from the second to the first volume, and ends upon altering the flow a second time such that the fluid is transported again from the first to the second volume.

While FIGS. 1A-1F illustrate a system in which the first and second volumes are located within separate containers, it should be understood that exchange of catalyst between first and second volumes can also be achieved when the first and second volumes are located within a single container. For example, in the set of embodiments illustrated in FIGS. 2A-2B, catalyst can be immobilized within volume 112, and fluid can be transported in the direction of arrow 120 and from first volume 112 to second volume 114. After at least a portion of the catalyst within first volume 112 is transported to and immobilized within second volume 114, the flow can be altered such that the fluid is transported in the direction of arrow 122 and from second volume 114 to first volume 112. As the fluid flows, at least a portion of the catalyst immobilized within second volume 114 can be entrained by the fluid. This catalyst can then be transported to and immobilized within first volume 112.

Fluid flow can be altered using any suitable instrumentation. For example, in the set of embodiments illustrated in FIGS. 1A-1F, optional valve 30 (e.g., a four-way valve) can be used to switch the direction of fluid flow between the first and second volumes. In FIGS. 1A-1C, valve 30 is oriented in a first position such that fluid from inlet 32 is transported to first volume 12, then to second volume 14, and finally through outlet 34. In FIGS. 1D-1F, the orientation of valve 30 has been altered such that fluid from inlet 32 is transported to second volume 14, then to first volume 12, and finally through outlet 34. Similarly, in the set of embodiments illustrated in FIG. 2A, optional valve 130 can be used to alter the flow path of the fluid.

In some embodiments, after altering the flow of the fluid, the fluid is transported in a different direction (e.g., substantially the opposite direction) across at least one of the first and second portions of support material, relative to the direction of flow prior to altering the path of the fluid. For example, in the set of embodiments illustrated in FIGS. 1A-1F and FIG. 2A, after the flow path is altered, the fluid is flowed in substantially the opposite direction across the first and second volumes (and, therefore, the first and second portions of support material).

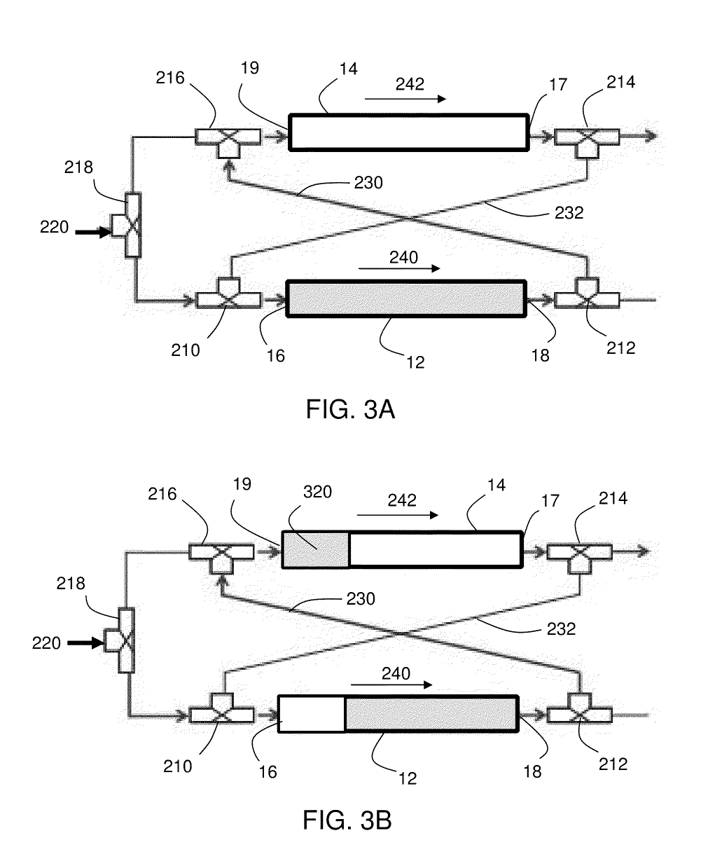

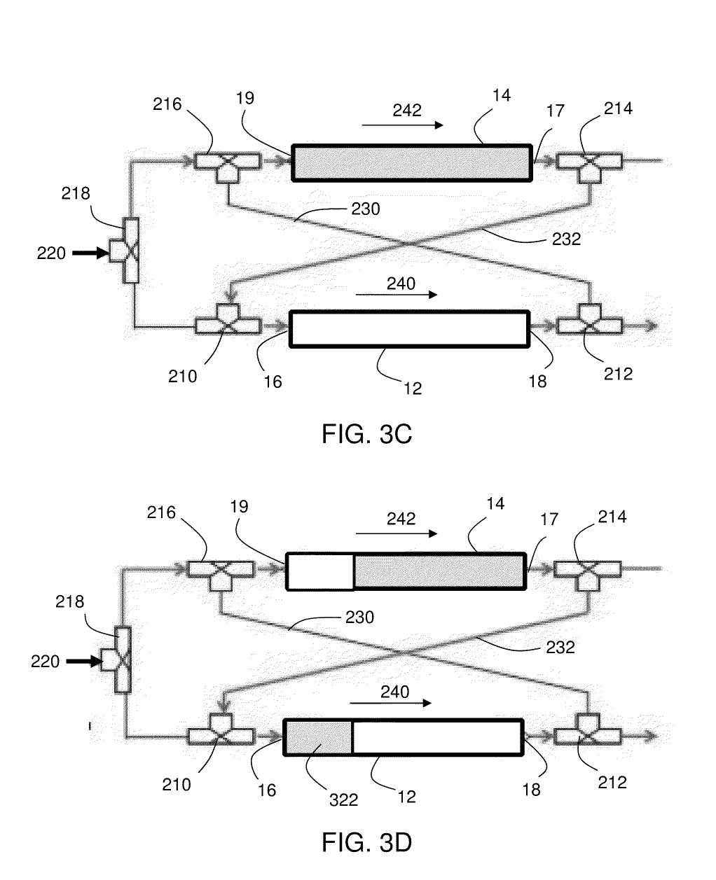

In some instances, after the flow of the fluid is altered, the fluid can be transported in substantially the same direction across at least one of the first and second portions of support material, relative to the direction of flow prior to altering the path of the fluid. For example, FIGS. 3A-3D include schematic illustrations of a system which incorporates a plurality of three-way valves that allow the direction of flow of the fluid across the first and second volumes (and, hence, the first and second portions of support material) to remain substantially constant after the flow is altered (i.e., the flow is changed such that the fluid flows first through the second volume and subsequently through the first volume). In FIGS. 3A-3D, interfaces 16 and 19 of volumes 12 and 14, respectively, serve as the inlets, and interfaces 18 and 17 of volumes 12 and 14, respectively, serve as the outlets. In this set of embodiments, three-way valve 210 is in direct fluidic communication with interface 16 (inlet), and three-way valve 212 is in direct fluid communication with interface 18 (outlet). In addition, three-way valve 214 is in direct fluidic communication with interface 17 (outlet), and three-way valve 216 is in direct fluid communication with interface 19 (inlet). Three way-valve 218, which directs the flow of incoming fluid stream 220, is in direct fluidic communication with valves 210 and 216. Three-way valves are also used to cross-connect the inlets and outlets of the volumes, with valve 216 in direct fluid communication with valve 212 via conduit 230, and valve 210 in direct fluid communication with valve 214 via conduit 232.

As illustrated in FIG. 3A, when the three-way valves are maintained in a first position, the inlet fluid in stream 220 can be transported through first volume 12 in the direction of arrow 240. As the fluid exits first volume 12 via interface 18, it enters valve 212 and is redirected to interface 19 of second volume 14 via valve 216. The fluid is then transported through second volume 14 in the direction of arrow 242. Catalyst can be entrained in the fluid as it passed through the first volume, transported out of the first volume, and immobilized in the second volume, as illustrated by the shaded region 320 in FIG. 3B.

In FIG. 3C, each of the valves (210, 212, 214, 216, and 218) have been switched to a second position. When the valves have been switched to these positions, the inlet fluid in stream 220 is transported through second volume 14 in the direction of arrow 242. As the fluid exits second volume 14 via interface 17, it enters valve 214 and is redirected to interface 16 of first volume 12 via valve 210. The fluid is then transported through first volume 12 in the direction of arrow 240. Catalyst can be entrained in the fluid, transported out of the second volume, and immobilized in the first volume, as illustrated by the shaded region 322 in FIG. 3D. In this way, catalyst can be exchanged back and forth between the first and second volumes.

Any suitable number of volumes can be used in the embodiments described herein. The system can include, in some embodiments, a third volume, a fourth volume, a fifth volume, or more (e.g., in a third container, fourth container, fifth container, etc.) in fluid communication with the first and second volumes. The additional volumes can be used to immobilize any catalyst that is transported out of the second volume upon the flow of a fluid. For example, in the set of embodiments illustrated in FIGS. 1A-1F, a third volume (not shown) can be positioned such that it is in direct fluid communication with second volume 14 (and, optionally, additional volumes can be positioned such that they are in fluid communication with the first three volumes). As fluid is transported through volume 12 and subsequently volume 14, catalyst entrained in the fluid exiting volume 14 can be immobilized within the third (or more) volumes. After a period of flow in a first direction, the flow can be altered such that the fluid first passes through the third volume (optionally, after passing through any additional volumes) before being transported through the second and subsequently the first volume. In some embodiments, the system includes at least 3, at least 4, at least 5, at least 10, at least 100, or at least 1000 volumes.

In some cases, the support material within the first and/or second (or more) volumes can be exposed to one or more conditions that enhance the level to which catalyst entrained in the fluid is immobilized via interactions with available sites within the support material in the volumes. For example, in some cases, the temperature of the support material in a downstream portion can be changed (e.g., increased or decreased) such that immobilization (e.g., re-adsorption) of catalyst is enhanced. In some cases, the fluid stream can be diluted (e.g., via an increase in fluid flow rate, via a decrease in the flow rate of a component that dissolves or otherwise entrains catalyst, etc.) such that the immobilization of the catalyst is enhanced. In some cases, the ionic strength of the fluid stream can be changed (e.g., via the addition of brine) such that the immobilization of the catalyst is enhanced. In some cases, the composition of the fluid stream can be changed (e.g., addition of an antisolvent) such that the immobilization of the catalyst is enhanced.

The term “catalyst,” as used herein, is given its ordinary meaning in the art, and refers to a material that is not substantially consumed during a chemical reaction and, when exposed to a set of conditions selected to cause a chemical reaction, either enables a chemical reaction that would otherwise not occur in the absence of the catalyst under essentially identical conditions, or increases the rate of reaction relative to the rate that would be observed under essentially identical conditions but without the catalyst. The embodiments described herein can employ any suitable type of catalyst. In some embodiments, the catalyst can include a metal (e.g., a transition metal catalyst, or any other suitable metal). In this context, a “metal” refers to a metallic material that is not covalently bound to a material other than the support material, as opposed to a metallic element that is bonded to a more electronegative element, as might be observed, for example, in a metal oxide. In some cases, the metal may not be covalently bonded to any other material (including the support material). In some cases, the catalyst can be a substantially pure metal (e.g., a substantially pure transition metal, or any other suitable metal).

The catalyst can be of any suitable phase (e.g., solid, liquid, etc.). In some embodiments, the catalyst can comprise an ionic liquid.

In some embodiments, the catalyst can include an organometallic molecule (i.e., a catalyst including a metallic component and an organic component). Suitable organometallic catalysts include, but are not limited to, triphenylphosphine palladium, Co(III) salen, Hoveyda-Grubbs catalyst, and Wilkonsen's catalyst. In some cases, the catalyst can include an organometallic molecule that has been modified to enhance immobilization (e.g., triphenylphosphine palladium sulphonate). In some cases, the catalyst can include an organic molecule such as, for example, enzymes, proline, 2,2,6,6-tetramethylpiperidine-1-oxyl, and 4-dimethylaminopyridine (DMAP). In some cases, the catalyst can include an organic molecule that has been modified to enhance selectivity (e.g. DMAP covalently bound to a polymer). One of ordinary skill in the art would be capable of selecting an appropriate catalyst for a given reaction system by, for example, performing a screening test in which a property of the reaction (e.g., reaction rate, reaction product, reaction by-product, etc.) in a system is measured and compared to the same property of one or more other systems to achieve a desired output.

The term “support material,” as used herein, refers to a material that performs to a lesser extent relative to the catalyst, and/or does not participate in the chemical reaction to be performed in the system of interest, and interacts with the catalyst such that, upon flowing a fluid through the system, the mobility of the catalyst is reduced relative to the mobility of the catalyst that would be observed under essentially identical conditions (e.g., flow rates, temperature, pressure, etc.) but in the absence of the support material. Any suitable type of support material can be used in the embodiments described herein. In some embodiments, the support material can comprise a metal oxide and/or a metalloid oxide. In some cases, the support material can include silica (e.g., silica gel, silica aerogel, glass, fumed silica, colloidal silica, and the like), alumina, alumina-silicate, other ceramics, polymers, and the like. In some cases, the support material can include a zeolite. In some cases, the support material can have a relatively high external surface area over which catalyst can be immobilized. For example, in some cases, the support material can be a porous solid (e.g., a foam, a collection of porous beads), a woven fabric, a membrane, and the like.

The catalyst can be immobilized on the support material via any suitable interaction. In some embodiments, the catalyst can be covalently bound to the support material. In some cases, the catalyst can be immobilized on the support material via noncovalent interactions. For example, the catalyst and the support material can interact via ionic bonds, hydrophobic interactions, hydrogen bonds, Van der Waals forces (i.e. “London dispersion forces”), or dipole-dipole bonds.

In some cases, the catalyst and/or support material can be modified to enhance the interaction between the support material and the catalyst. For example, in some cases, the catalyst and/or support material can comprise (e.g., it can be modified to comprise) a fluorous compound (i.e., a compound comprising a fluorine atom). In some cases, the support material can be modified to be hydrophilic or hydrophobic to enhance the interaction with a hydrophilic or hydrophobic catalyst, respectively.

As used herein, the term “fluid” generally refers to a substance that tends to flow and to conform to the outline of its container on the time frame of reactions described herein. Typically, fluids are materials that are unable to withstand a static shear stress, and when a shear stress is applied, the fluid experiences a continuing and permanent distortion. The fluid may have any suitable viscosity that permits at least some flow of the fluid. In some embodiments, the fluid can comprise a liquid or a gas. Other non-limiting examples of fluids include supercritical fluids, free-flowing solid particles (e.g., colloids, vesicles, etc.), viscoelastic fluids, and the like. As mentioned above, in some embodiments, the fluids described herein contain one or more reactants that can be flowed through one or more volumes to perform a catalytic chemical and/or biological reaction.

Any suitable type of container can be used in association with the embodiments described here. In some cases, the support material can be contained within a reactor (e.g., a quartz tube reactor, metal reactor, etc.). In some embodiments, the catalyst can form a packed bed within the container. In addition, the support material container can be any suitable size. In some cases, the container can be at least about 0.001 liters, at least about 0.01 liters, at least about 0.1 liters, at least about 1 liter, at least about 10 liters, at least about 100 liters, at least about 1000 liters, or larger. In some cases, the container can be a microreactor. For example, the container can have a volume of less than about 100,000 microliters, less than about 10,000 microliters, less than about 1000 microliters, less than about 100 microliters, less than about 10 microliters, or smaller.

In some, but not all embodiments, some or all components of the systems and methods described herein are microfluidic. “Microfluidic,” as used herein, refers to a device, apparatus or system including at least one fluid channel having a largest cross-sectional dimension of less than about 1 mm, and a ratio of length to largest cross-sectional dimension perpendicular to the channel of at least 3:1. As used herein, the “cross-sectional dimension” of a channel is measured perpendicular to the direction of fluid flow. A “microfluidic channel” or a “microchannel” as used herein, is a channel meeting these criteria. In one set of embodiments, all fluid channels containing embodiments of the invention are microfluidic.

A “channel,” as used herein, means a feature on or in an article (substrate) that at least partially directs the flow of a fluid. The channel can have any cross-sectional shape (circular, oval, triangular, irregular, square or rectangular, or the like) and can be covered or uncovered. In embodiments where it is completely covered, at least one portion of the channel can have a cross-section that is completely enclosed, or the entire channel may be completely enclosed along its entire length with the exception of its inlet(s) and outlet(s). A channel may also have an aspect ratio (length to average cross sectional dimension) of at least 2:1, more typically at least 3:1, 5:1, or 10:1 or more. In some cases the dimensions of the channel may be chosen such that fluid is able to freely flow through the article or substrate. The dimensions of the channel may also be chosen, in some cases, to allow a certain volumetric or linear flow rate of fluid in the channel. In some embodiments, the length of the channel may be selected such that the residence times of the fluid at a predetermined flow rate is sufficient to achieve a desired rate of reaction within the channel Of course, the number of channels and the shape of the channels can be varied by any method known to those of ordinary skill in the art. In some cases, more than one channel may be used.

A variety of materials and methods, according to certain aspects of the invention, can be used to form systems, including microfluidic systems, described herein. For example, in some embodiments, the containers within which the support material is positioned may comprise tubing such as, for example, flexible tubes (e.g., PEEK tubing), capillary tubes (e.g., glass capillary tubes), and the like. In some embodiments, various components can be formed from solid materials, in which channels (e.g., microfluidic channels) can be formed via micromachining, film deposition processes such as spin coating and chemical vapor deposition, laser fabrication, photolithographic techniques, etching methods including wet chemical or plasma processes, and the like. See, for example, Scientific American, 248:44-55, 1983 (Angell, et al). In one set of embodiments, at least a portion of the fluidic system is formed of silicon by etching features in a silicon chip. Technologies for precise and efficient fabrication of various fluidic systems and devices of the invention from silicon are known. In another embodiment, various components of the systems and devices of the invention can be formed of a polymer, for example, an elastomeric polymer such as polydimethylsiloxane (“PDMS”), polytetrafluoroethylene (“PTFE” or Teflon®), or the like. In some cases, various components of the system may be formed in other materials such as metal, ceramic, glass, Pyrex®, etc.

Different components can be fabricated of different materials. For example, a base portion including a bottom wall and side walls of a container in which support material is located can be fabricated from a transparent or at least partially transparent material, such as glass or a transparent polymer, for observation and/or control of the fluidic process (e.g., a reaction within the container), and a top portion can be fabricated from an opaque material such as silicon. Components of the system can be, in some instances, coated so as to expose a desired chemical functionality to fluids that contact interior channel walls, where the base supporting material does not have a precise, desired functionality. For example, components can be fabricated as illustrated, with interior channel walls coated with another material. Material used to fabricate various components of the systems and devices of the invention, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device.

In one set of embodiments, various components of the invention can be fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one set of embodiments, the hardenable fluid comprises a polymeric liquid or a liquid polymeric precursor (i.e. a “prepolymer”). Suitable polymeric liquids can include, for example, thermoplastic polymers, thermoset polymers, or mixture of such polymers heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, etc.

Silicone polymers can be used in one set of embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS exhibit several properties that can be beneficial in simplifying fabrication of structures (e.g., microfluidic containers, etc.) of the invention. For instance, such materials can be relatively inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric, and thus may be useful for forming very small features with relatively high aspect ratios, which can be useful in certain embodiments of the invention. Flexible (e.g., elastomeric) molds or masters can be advantageous in this regard.

One advantage of forming structures (e.g., microfluidic structures) from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, components can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy, et al.), incorporated herein by reference.

As used herein, the following elements are considered to be metallic elements: lithium, sodium, potassium, rubidium, cesium, francium, beryllium, magnesium, calcium, strontium, barium, radium, zinc, molybdenum, cadmium, scandium, titanium, vanadium, chromium, manganese, iron, cobalt, nickel, copper, yttrium, zirconium, niobium, technetium, ruthenium, rhodium, palladium, silver, hafnium, tantalum, tungsten, rhenium, osmium, iridium, platinum, gold, mercury, rutherfordium, dubnium, seaborgium, bohrium, hassium, meitnerium, darmstadtium, roentgenium, ununbium, aluminium, gallium, indium, tin, thallium, lead, bismuth, ununtrium, ununquadium, ununpentium, ununhexium, lanthanum, cerium, praseodymium, neodymium, promethium, samarium, europium, gadolinium, terbium, dysprosium, holmium, erbium, thulium, ytterbium, lutetium, actinium, thorium, protactinium, uranium, neptunium, plutonium, americium, curium, berkelium, californium, einsteinium, fermium, mendelevium, nobelium, and lawrencium.

U.S. Provisional Patent Application No. 61/297,504, filed Jan. 22, 2010, and entitled “Trapping, Recycling, and Other Techniques Involving Catalysts,” is incorporated herein by reference in its entirety for all purposes. All other patents and patent publications mentioned herein are also incorporated by reference in their entirety for all purposes.

The following example is intended to illustrate certain embodiments of the present invention, but does not exemplify the full scope of the invention.

### EXAMPLE

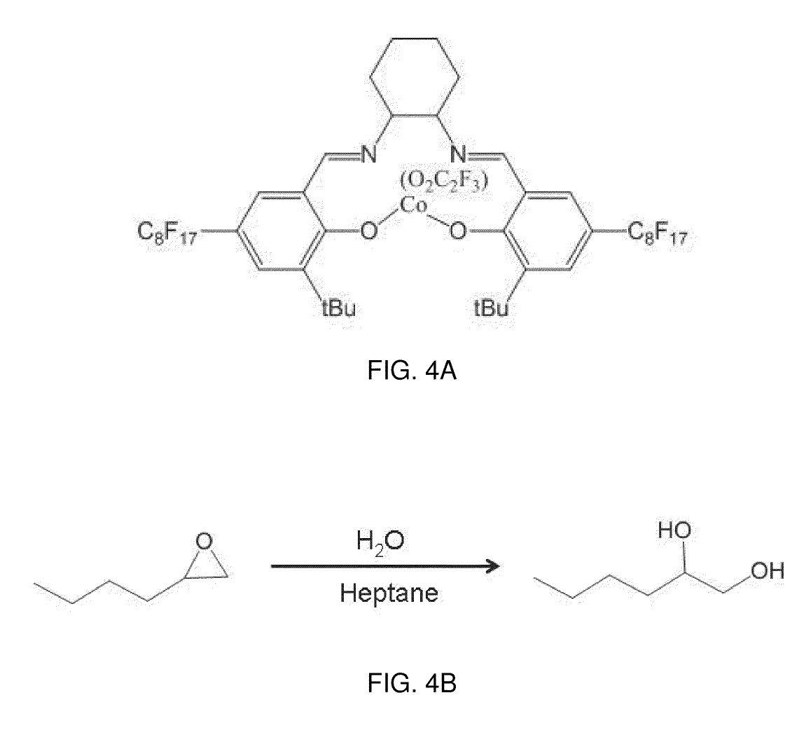

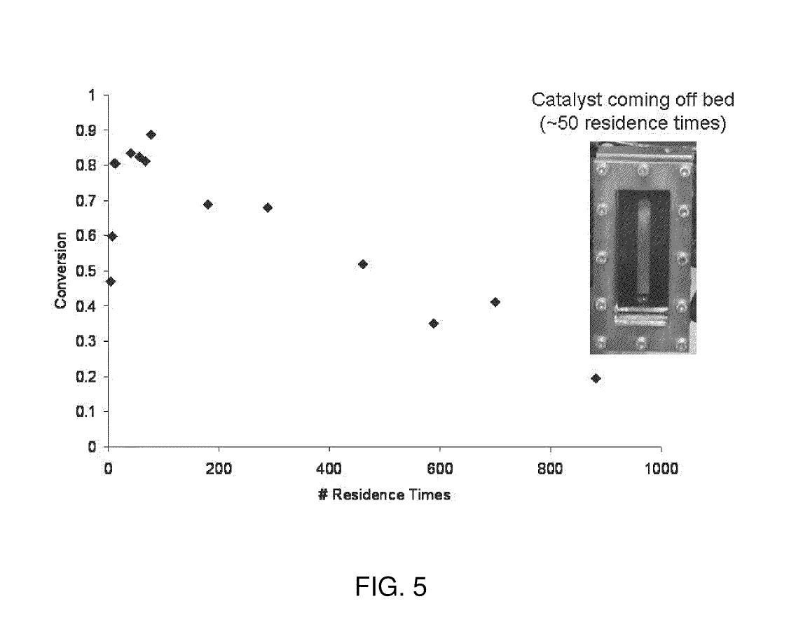

This example describes the use of multiple bed portions to control the leaching of catalyst in a system for performing catalytic reactions. About 40 mg of FluoroFlash (Fluorous Technologies, Inc.) silica gel was loaded into a 4 mm wide×28 mm long×600 micron deep silicon nitride coated silicon microreactor with about 40 microliters of void space using ethanol as a carrier solvent. The silica gel consisted of semi-spherical particles with a mean diameter of about 67 microns containing 27 wt % organic (carbon, hydrogen, and fluorine) material, and had about 430 m²/g of surface area and about 6 nm pore diameters. The bed was dried by passing pressurized nitrogen, 10 psig, through the reactor for 15 minutes. The bed was connected, in series, to about 5 microliters of 0.02″ Teflon tubing, a 20 psi back pressure regulator (Upchurch Scientific, P-791), and then about 10 microliters of 0.02″ inner diameter Teflon tubing. Racemic fluorous-tagged Co(III) Salen catalyst (FIG. 4A), 12 mg, was dissolved in tetrahydrofuran, 0.4 mL, and manually passed over the bed. The bed was stored at 4° C. for 16 hours, after which it was washed with hexanes, 3 mL. A syringe pump (Harvard Apparatus, PHD-2000) was used to flow epoxyhexane (1.0 M) in equal volumes of heptane and water through the bed with a total average flowrate of 10 microliters per minute for 75 hours at room temperature. Samples of the eluent were periodically taken and analyzed for the conversion of epoxyhexane to hexane diol (FIG. 4B) using gas chromatography. Conversion decreased from 83% to 20% over the course of the 75 hours with a single bed (FIG. 5). This decrease in conversion was likely due to elution of the catalyst, as indicated by discoloration of the organic phase.

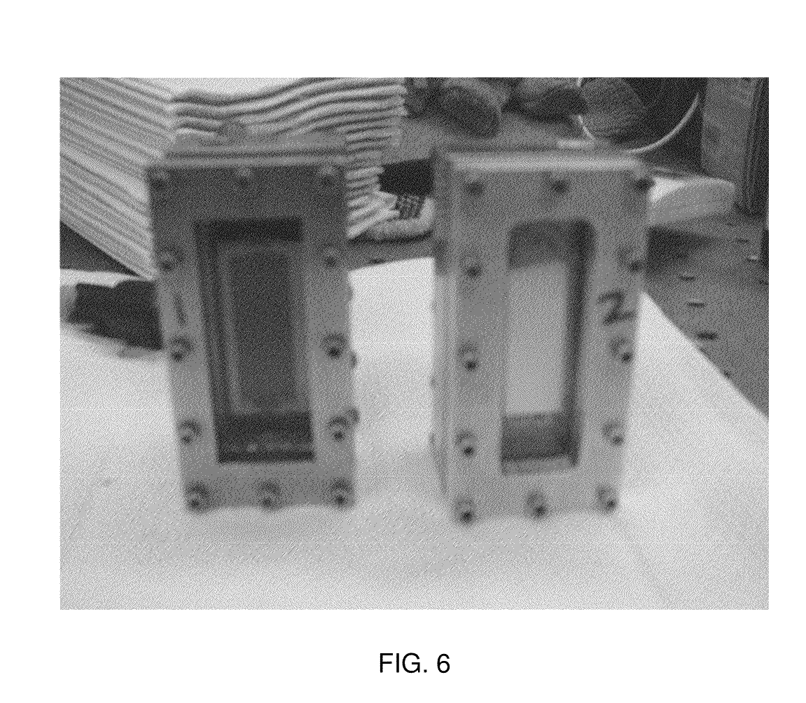

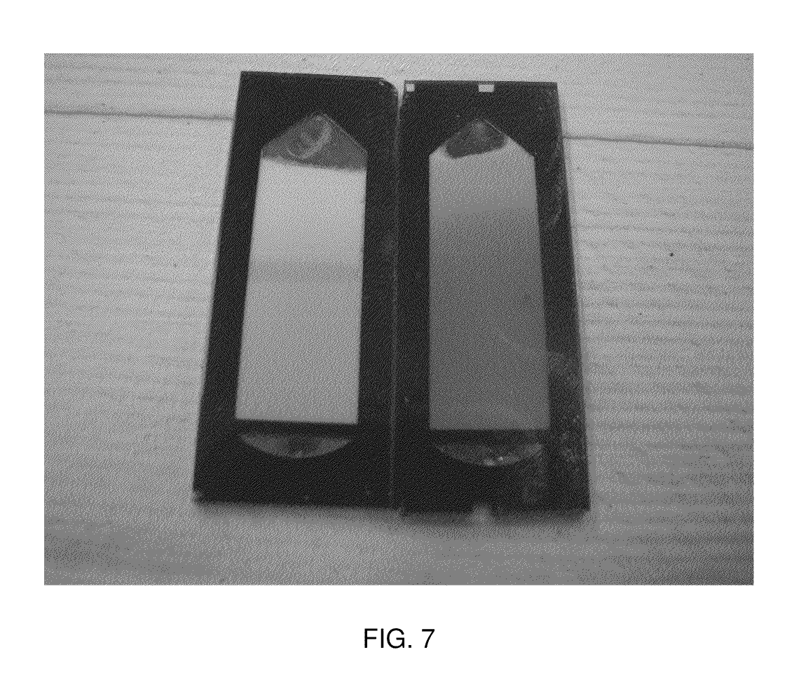

This experiment was repeated using a second bed to aid in catalyst recapture. Two silicon nitride coated silicon microreactors, 10 mm wide×26 mm long×600 micron deep, each with about 100 microliters of void space, were used in this experiment. Both silicon microreactors were loaded with about 100 mg of FluoroFlash silica gel. The first silicon microreactor bed was loaded with racemic fluorous-tagged Co(III) Salen catalyst, 30 mg, in 1 mL, and the second reactor was left unloaded (FIG. 6). The reactors were then connected in series using 0.02″ inner diameter Teflon tubing. A biphasic mixture of epoxyhexane (1 molar) in a 1:1 volumetric ratio mixture of heptane and water was fed to the first bed (and subsequently, to the second bed) at a total flowrate of 20 microliters/min for 24 hours at room temperature. After 24 hours of operation, the first bed was heated to 50° C. and washed by feeding, to the first bed and subsequently the second bed, 10 mL of tetrahydrofuran followed by 5 mL of methanol to speed the transfer of the catalyst from the first bed to the second bed. After the wash step, the first bed was colorless and the second bed was colored (FIG. 7).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A system, comprising:
a first portion of support material for immobilizing a catalyst, the first portion of support material and any catalyst thereon together defining a first volume, the first volume containing a catalyst at a first ratio of moles of catalyst to mass of the first portion of support material; and
a second portion of support material for immobilizing a catalyst, the second portion of support material and any catalyst thereon together defining a second volume, the second portion of support material in fluid communication with the first portion of support material, the second volume containing the catalyst at a second ratio of moles of catalyst to mass of the second portion of support material that is smaller than the first ratio.

2. A method, comprising:
establishing the flow of a fluid over a first portion of support material for immobilizing a catalyst, the first portion of support material and any catalyst thereon together defining a first volume having a first ratio of moles of catalyst to mass of support material; and
subsequently establishing the flow of the fluid over a second portion of the support material in fluid communication with the first portion, the second portion of support material and any catalyst thereon together defining a second volume having a second ratio of moles of catalyst to mass of support material,
wherein the second ratio is smaller than the first ratio.

3. A system as in claim 1, wherein the first volume occupies at least about 10% of the area of a cross-section of a container in which the volume is located.

4. A method as in claim 2, wherein, upon establishing the flow of the fluid over the first portion of support material, at least a portion of the catalyst is transported out of the first volume.

5. A method as in claim 4, wherein, upon establishing the flow of the fluid over the second portion of support material, at least a portion of the catalyst transported out of the first volume is immobilized on the second portion of support material.

6. A method as in claim 1, further comprising altering a path of fluid supplied to the first and second volumes such that the fluid supplied to the first and second volumes flows over the first portion of support material subsequent to flowing over the second portion of support material.

7. A method as in claim 6, wherein, subsequent to altering the path of fluid supplied to the first and second volumes, a portion of the catalyst within the second portion of support material is transported out of the second volume.

8. A method as in claim 6, wherein, subsequent to altering the path of fluid supplied to the first and second volumes, a portion of the catalyst transported out of the second volume is immobilized on the first portion of support material.

9. A method as in claim 6, wherein, subsequent to altering the path of fluid supplied to the first and second volumes, fluid is transported in substantially the same direction across at least one of the first and second portions of support material, relative to the direction of flow prior to altering the path of the fluid.

10. A method as in claim 6, wherein, subsequent to altering the path of fluid supplied to the first and second volumes, fluid is transported in substantially the opposite direction across at least one of the first and second portions of support material, relative to the direction of flow prior to altering the path of the fluid.

11. A system as in claim 1, wherein the first and second portions of support material are located within a single container.

12. A system as in claim 1, wherein the first and second portions of support material are located within separate containers.

13. A system as in claim 1, wherein the second ratio of moles of catalyst to mass of support material is at least about 5 times smaller than the first ratio of moles of catalyst to mass of support material.

14. A system in claim 1, wherein substantially no catalyst is present within the second volume.

15. A system as in claim 1, wherein the catalyst is covalently bound to the first and/or second portion of support material.

16. A system as in claim 1, wherein the catalyst is non-covalently bound to the first and/or second portion of support material.

17. A system as in claim 1, wherein the first and/or second portion of support material is located within a reactor.

18. A system as in claim 17, wherein the reactor comprises a packed-bed reactor.

19. A system as in claim 1, wherein the support material comprises a metal oxide, a metalloid oxide, and/or a polymer.

20. A system as in claim 1, wherein the support material comprises alumina, silica, and/or silicate.

21. A system as in claim 1, wherein the catalyst comprises a metal.

22. A system as in claim 1, wherein the catalyst comprises an enzyme.

23. A system as in claim 1, wherein the catalyst comprises fluorine.

24. A system as in claim 1, wherein the catalyst comprises an ionic liquid.

25. A system as in claim 1, wherein the fluid comprises a liquid.

26. A system as in claim 1, wherein the fluid comprises an ionic liquid.

27. A system as in claim 1, wherein the fluid comprises a gas.

28. A system as in claim 1, wherein the fluid comprises a solvent.

29. A system as in claim 1, wherein the fluid comprises an inorganic component.

30. A system as in claim 1, wherein the fluid comprises an organic component.
